SEQUENCE LISTING (6 sequences)

SEQ ID NO: 1 - protein, 17 aa, Acidivorax temperans H+
IIANDNALGAGLAFYAA

SEQ ID NO: 2 - protein, 12 aa, Acidivorax temperans H+
  misc_feature (12)..(12): “X” represents an amino acid of unknown identity
YPNQFQVYSPYX

SEQ ID NO: 3 - protein, 8 aa, Acidivorax temperans H-
  misc_feature (6)..(8): “X” represents an amino acid of unknown identity
EVEVLXYX

SEQ ID NO: 4 - protein, 12 aa, Acidivorax temperans H-
AAGLIPVAHGSQ

SEQ ID NO: 5 - DNA, 1405 bp, Acidivorax temperans
tgccttacacatgcaagtcgaacggtaacaggtcttcggatgctgacgagtggcgaacgggtgagtaatacatcggaacgtgcccgatcgtgggggataacgaagcgaaagctttgctaataccgcatacgatctacggatgaaagcaggggaccgcaaggccttgcgcgaacggagcggccgatggcagattaggtagttggtgggataaaagcttaccaagccgacgatctgtagctggtctgagaggacgaccagccacactgggactgagacacggcccagactcctacgggaggcagcagtggggaattttggacaatgggcgaaagcctgatccagccatgccgcgtgcaggatgaaggccttcgggttgtaaactgcttttgtacggaacgaaaagactctggttaatacctggggtccatgacggtaccgtaagaataagcaccggctaactacgtgccagcagccgcggtaatacgtagggtgcaagcgttaatcggaattactgggcgtaaagcgtgcgcaggcggttatataagacagatgtgaaatccccgggctcaacctgggaactgcatttgtgactgtatagctagagtacggcagagggggatggaattccgcgtgtagcagtgaaatgcgtagatatgcggaggaacaccgatggcgaaggcaatcccctgggcctgtactgacgctcatgcacgaaagcgtggggagcaaacaggattagataccctggtagtccacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaatcctttagagatagaggagtgctcgaaagagagccgtaacacaggtgctgcatggctgtcgtcagctcgtgtcgtgagatgttgggttaagtcccgcaacgagcgcaacccttgccattagttgctacgaaagggcactctaatgggactgccggtgacaaaccggaggaaggtggggatgacgtcaagtcctcatggcccttataggtggggctacacacgtcatacaatggctggtacagagggttgccaacccgcgagggggagctaatcccataaagccagtcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgc

SEQ ID NO: 6 - protein, 12 aa, Acidovorax temperans
  MISC_FEATURE (12)..(12): “X” represents an amino acid of unknown identity
YPNQFQVYNPYX

(19) United States
(12) Patent Application Publication
(10) Pub. No.: US 2006/0030021 A1
(43) Pub. Date: Feb. 9, 2006

(54) Novel microorganism acidovorax temperans and uses therefor

(76) Inventor: Susan Jane Turner, Auckland (NZ)

Correspondence Address: FISH & RICHARDSON PC, P.O. BOX 1022, MINNEAPOLIS, MN, 55440-1022, US

(21) Appl. No.: 10/515,311

(22) PCT Filed: May 22, 2003
(86) PCT No.: PCT/NZ03/00100

§ 371 Date: Jun. 17, 2005

(30) Foreign Application Priority Data
May 22, 2002 (NZ) 519137

(51) Int. Cl. C12N 1/20; C02F 3/34

(52) U.S. Cl. 435/252.1; 435/262; 210/601

(57) ABSTRACT

Novel strains of Acidovorax temperans are described. The novel characteristics of these strains allow their use in promoting flocculation or biofilm formation in industrial systems, as well as in predicting the ability of such systems to form floc or biofilm.

### TECHNICAL FIELD

The invention relates to novel micro-organisms and uses therefore. The invention has particular application in methods of water treatment.

### BACKGROUND

In industrial processes such as wastewater treatment and other large scale processes involving the bulk storage and transfer of large volumes of aqueous suspensions, bacteria are invariably present. These bacteria may exist in suspension as single cells or they may aggregate to form flocs that are often large enough to be visible and settle under the influence of gravity. Bacteria also adhere to surfaces forming multi-layered biofilms that coat the insides of vessels and pipes. In both the formation of flocs and biofilms, bacterial cells interact and communicate through a series poorly understood processes sometimes referred to as quorum sensing.

Activated sludge is a large scale bioprocess technology that is in widespread use for the treatment of domestic and industrial waste waters. The principal objective of the treatment process is to reduce the suspended solids content and biological oxygen demand (BOD) of the wastewater to a level which is suitable for further treatment, such as UV disinfection, and/or subsequent discharge into the receiving environment. The process involves the prior maceration of solids to produce a concentrated sewage suspension followed by the vigorous aeration of the mixture to promote the growth of microorganisms, principally heterotrophic bacteria. During this process nutrients present in the suspension are converted to microbial biomass and in some circumstances there is complete mineralisation to inorganic nutrients and gases (eg nitrogen). (Davey and O'Toole, 2000)

The biomass (floc) formed in the process of activated sludge treatment is removed by physical means (usually by settling under gravity, or alternatively by DAF (desolved air floatation)) and the resulting effluent is discharged to the environment, often following a further disinfection step. The settled floc is dewatered by compression or by centrifugation. In this form, the floc is known as sludge and is disposed of by incineration, landfill or in some cases by further anaerobic digestion to produce methane.

A key step in the biological processes involved in water treatment is the aggregation of microbial cells to form settleable flocs which enables the biomass to be separated from the liquid. If aggregation and floc formation are inefficient, then solids are carried over into the effluent. Major separation problems can occur if the microbial community becomes dominated by filamentous or other microorganisms that hold the solids in a gel-like suspension. In this situation (known as “bulking”), the separation of the biomass from the water becomes either infeasible or very difficult

Fixed growth reactors are another widely-used water treatment process in which organic and inorganic nutrients are removed by conversion to microbial biomass. In these systems the microbial community is attached to a surface over which the wastewater flows. The formation of stable biofilms is therefore important in this process because it is the presence of the biofilm which both utilises the nutrients and enables the process to be managed in a consistent and predictable fashion.

The ability to control, manage and/or predict floc formation is important to the operational success of wastewater treatment systems based on the activated sludge process. The ability to control, manage and/or predict biofilm formation is important for the success of water treatment systems utilising fixed-film reactors and also for the prevention and treatment of industrial fouling.

### SUMMARY OF THE INVENTION

In a first aspect, the invention provides a bacteria (designated Acidovorax temperans H+) capable of being isolated from activated sludge and which is capable of forming biofilms and has one or more of the following characteristics: Colonies of a rough form exhibiting a creamy yellow colour, having a raised profile, and being approximately 1.5-2.5 mm in diameter; Gram negative; Rod-shaped cells; Intracellular presence of Poly B-hydroxybutyrate; Mega-pili on the surface; Paracrystalline surface layers; Sensitivity to growth in the presence of NaCl; and, having a 16S r RNA sequence of SEQ ID NO.5, or variant thereof.

Preferably, Acidovorax temperans H+ has all of the above characteristics.

In a second aspect, the invention provides a bacteria (designated Acidovorax temperans H−) capable of being isolated from activated sludge and which is capable of flocculating and has one or more of the following characteristics: Colonies having a smooth margin, exhibiting a creamy yellow colour, having a raised profile, and being approximately 1.5-2.5 mm in diameter; Gram negative; Rod-shaped cells; Intracellular presence of Poly B-hydroxybutyrate; Mega-pili on the surface; Paracrystalline surface layers; Sensitivity to growth in the presence of NaCl; and, having a 16S r RNA sequence of SEQ ID NO.5, or variant thereof.

Preferably, Acidovorax temperans H− exhibits all of the above characteristics.

In related aspects, the invention provides cultures of Acidovorax temperans having the phenotype H+, and/or Acidovorax temperans having the phenotype H−.

In a third aspect, the invention provides a method of promoting the formation of a biofilm in a composition, the method including at least the step of adding to the composition Acidovorax temperans H+.

In a fourth aspect, the invention provides a method of promoting flocculation of bacteria in a composition, the method including at least the step of adding to the composition Acidovorax temperans H−.

In a fifth aspect, the invention provides a method of water treatment, the method including at least the step of adding to a water treatment system either or both of Acidovorax temperans H+ and Acidovorax temperans H−.

Preferably, Acidovorax temperans H− is added where the promotion of flocculation of bacteria is desired. Alternatively, Acidovorax temperans H+ is added where promotion of biofilm formation is desired.

Preferably, Acidovorax temperans H+ and/or H− are added to activated sludge within the water treatment system.

Preferably, Acidovorax temperans H+ and/or Acidovorax temperans H− are added to the water treatment system as liquid cultures.

In a sixth aspect, the invention provides a method of water treatment, the method including at least the steps of: (a) detecting the presence of one or both of Acidovorax temperans H+ and Acidovorax temperans H− in a water treatment system; and dependent on the results, (b) adding to the treatment plant an amount of either or both of Acidovorax temperans H+ and Acidovorax temperans H−.

Preferably, the method of the sixth aspect further includes the step of determining the ratio of Acidovorax temperans H+ to Acidovorax temperans H− present in treatment system.

Preferably, Acidovorax temperans H− is added where promotion of flocculation of bacteria is desired. Alternatively Acidovorax temperans H+ is added where promotion of biofilm formation is desired.

Preferably, the bacteria are detected by standard microbiological techniques, including microscopy.

In a seventh aspect, the invention provides a method of predicting the likelihood of bacteria in a composition to flocculate, the method including at least the step of detecting the presence of Acidovorax temperans H− and/or Acidovorax temperans H+.

In an eighth aspect, the invention provides a method of predicting the likelihood of bacteria in a composition to form a biofilm, the method including at least the step of detecting the presence of Acidovorax temperans H+ and/or Acidovorax temperans H−.

Preferably a method of the seventh or eighth aspects involves detecting the presence of both Acidovorax temperans H+ and Acidovorax temperans H−. More preferably, the ratio of Acidovorax temperans H+ to Acidovorax temperans H− is determined, a high ratio being predictive of the likelihood of biofilm formation and a low ratio being predictive of the likelihood of bacteria to flocculate.

In a nineth aspect, the invention provides a method of maintaining viability of, and/or promoting growth of, Acidovorax temperans H+ and/or Acidovorax temperans H− in a composition, including at least the steps of: (a) determining the NaCl concentration in the composition; and, where the concentration is above approximately 0.5%, (b) reducing it to below approximately 0.5%.

In a related aspect, the invention provides a method of maintaining viability of, and/or promoting growth of, Acidovorax temperans H+ and/or Acidovorax temperans H− in a composition, including at least the steps of: (a) determining the NaCl concentration in the composition; and, where the concentration is above approximately 1%, (b) reducing it to below approximately 1%.

In yet a further aspect, the invention provides a water treatment composition, the composition including either or both of Acidovorax temperans H+ and Acidovorax temperans H−.

Preferably the composition is a liquid culture of either of Acidovorax temperans H+ or Acidovorax temperans H−.

### DRAWINGS

These and other aspects of the present invention, which should be considered in all its novel aspects, will become apparent from the following description, which is given by way of example only, with reference to the accompanying figures, in which:

FIG. 1 shows CB2 colonies on R2A agar, incubated at 28° C. CB2 colonies grown on R2A agar exhibit two morphologies; a rough-margined form (Panels A to B) that has a distinctive halo when viewed in oblique light (panel B) and a smooth entire-margined form with no halo (panel D). When viewed at 100× magnification, the margins of rough colonies appear as islands of clumped cells surrounded by patches of bare agar on which there are few cells (panel C). In contrast, the margins of smooth non-haloed colonies appear as a consolidated edge of tightly packed cells (panel E).

FIG. 2 shows biofilm formation by CB2 morphotypes. The ability of CB2 clls to attach to surfaces and initiate biofilm development was investigated using a crystal violet binding assay, Halo positive (H+) and halo-negative (H−) colonies were grown overnight in R2 broth, then subcultured into fresh media in microtitre trays. Replicate wells of the dish were rinsed and stained with crystal violate following incubation for 3, 21, and 45 hourse. A media-only control (−) indicates background staining due to chemical interactions. The intensity of crystal violet staining in the H+ inoculated wells is high after 3 hours indicating rapid attachment and retention of cells. Crystal violet retention by H− inoculated cells was initially poor and only exceeded background levels after 21 hours of incubation. Biofilms formed after 21 hours by H− are unstable and can be removed by vigorous washing, while those of H+ remain attached under the same treatment.

FIG. 3 shows flocculation and settling of CB2 broth cultures. The ability of H+ and H− broth cultures to flocculate and settle under gravity was evaluated by measuring the volume of floe in 10 ml pipettes over a 72 hour period (panel A). The lower and higher arrows indicate the height of the floc from H− and H+ cultures respectively, at the conclusion of the experiment H− cultures produced a compressed floc that settled to occupy less than 50% of the culture volume within 20 hours (Panel B). In contrast, H+ floe settled more slowly requiring in excess of 40 hours to reduce their volume by 50% and produced a less compressed floc at the end of the experiment.

FIG. 4 shows SDS PAGE of surface proteins of CB2 morphotypes. The surface-associated proteins of H+ and H− colonies, grown on R2A agar, were extracted by gentle heat treatment colony suspensions and resolved by denaturing SDS PAGE. The two colony morphotypes differed in the expression of three proteins (arrowed) that ranged in size from approximately 40 kDa to 45 kDa. M denotes molecular weight standard (kDa).

FIG. 5 shows transmission electron microscopy of CB2 surface structures. The cell surface morphology of H+ and H− cells was investigated by TEM of negatively stained colony suspensions. The surfaces of both H+ and H− cells were covered in thick appendages that comprised a tube-like structure with an internal flexible strand (panel D) and which appeared to form attachments between cells (panel E). H+ cells also appeared to be surrounded by long thin fibrils (arrowed in panel A) that extended out beyond a matrix of extracellular material that surrounded may cells (arrowed in panel B).

FIG. 6 A. shows CB2 partial 16S ribosomal RNA gene sequence (SEQ ID No. 5). The near-full length 16S sequence was determined from a DNA template amplified from CB2 genomic DNA using universal PCR primers that amplify an approximately 1500 bp fragment corresponding to positions numbering 8-1509 of the E. coli 16S gene (Brosius et al, 1981).

B. shows sequence comparison. Results (highest 20 scores) of a BLAST search with the 16S sequence for CB2 (Query sequence) against the GenBAnk database. The highest score (2761) was to GenBank entry AF078766 Acidovorax temperans.

C. shows homology of CB2 to A. temperans. Sequence alignment of CB2 (query sequence) against GenBank entry AF078766 Acidovorax temperans (subject sequence). The two sequences share 99% homology over a 1405 bp overlap.

FIG. 7 shows a TEM electron micrograph of negatively stained CB2H− cells showing a “mega-pilus” and also paracrystalline array structure of both the pilus sheath and bacterial cell surface. Cells were grown on R2A media plates.

FIG. 8 shows a TEM picture of negatively stained CB2- cells grown in Terrific broth. The bright dense spheres inside the cells are likely polyhyroxybutyrate granules.

### DETAILED DESCRIPTION OF THE INVENTION

The following is a description of the present invention, including preferred embodiments thereof, given in general terms. The invention is further elucidated from the disclosure given under the section “Examples” which provides experimental data supporting the invention and specific examples thereof.

The inventor has surprisingly identified two novel phenotypes of the bacteria Acidovorax temperans, herein referred to as H+ and H−, isolated from activated sludge. The inventor believes that the phenotypes observed are representative of two novel strains of Acidovorax temperans. The inventor has surprisingly discovered that these novel strains of bacteria have an affect on the ability of populations of bacteria, including mixed populations of bacteria, to flocculate and/or to form biofilms. In addition, the inventor has found that the presence and/or level of either or both of these strains of bacteria in a composition may be predictive of the ability of bacteria in such a composition to flocculate and/or form biofilms.

Acidovorax is a member of the beta Proteobacteria and a wider group of heterotrophic gram-negative bacteria that are commonly found in biofilms and wastewater flocs. Acidovorax temperans is a major component of the microbial flora found in activated sludge. The identification and characterisation of two new strains of Acidovorax temperans, namely H+ and H−, as detailed herein, may be used for the benefit of water treatment processes through the promotion of flocculation, or the improvement of other large scale industrial processes where inhibition or promotion of biofilm formation has not been recognised.

A first and second embodiment of the invention relates to the novel bacterial strains designated Acidovorax temperans H+ and H− and to cultures containing same. Cultures of these bacteria have been forwarded to the Australian Government Analytical Laboratories (AGAL), of 1 Suakin Street, Pymble, NSW 2073, Australia for deposit in accordance with the Budapest Treaty. The deposits have been allocated the accession numbers: A. temperans H+: NM03/35786; and, A. temperans H−: NM03/35787.

The novel bacteria of the invention have been isolated or purified. As used herein, the terms “isolated” or “purified” are intended to mean that the bacteria have been separated from an environment in which they naturally reside. The terms should not be taken to indicate the extent to which the bacteria may be purified.

In accordance with the invention, cultures of Acidovorax temperans H+ or H− are preferably “pure cultures”. This term is intended to refer to cultures which contain at least a predominance of Acidovorax temperans H+ or H−, most preferably only Acidovorax temperans H+ or H−.

A culture of the invention may be obtained by culturing bacteria following isolation from a mixed culture of bacteria, the environment in which they naturally reside, or any other composition containing them. In a preferred embodiment of the invention a culture is obtained by isolation of the bacteria from an activated sludge as described in further detail herein after under the heading “Examples”. Persons of general skill in the art to which the invention relates may appreciate alternative sources and means for isolating Acidovorax temperans H+ or H−. It may be possible to isolate Acidovorax temperans H+ or H− from human clinical samples.

Following isolation and/or culturing one or more of the following characteristics of H+ and H− can be used to confirm the identity of H+ and/or H−.

Colonies of substantially pure cultures of Acidovorax temperans H+ or H− of the invention exhibit a creamy yellow colour, have a raised profile and are approximately 1.5-2.5 mm in diameter, particularly when grown on R2A media (Difco Laboratories, Maryland, USA) at 28° C. for 48 hours.

Under light microscopy cells are observed to be Gram negative rods measuring approximately 0.5 μm×1.5-2.0 μm. Sudan Black staining of cells grown in Terrific broth with aeration indicates the intracellular presence of Poly B-hydroxybutyrate.

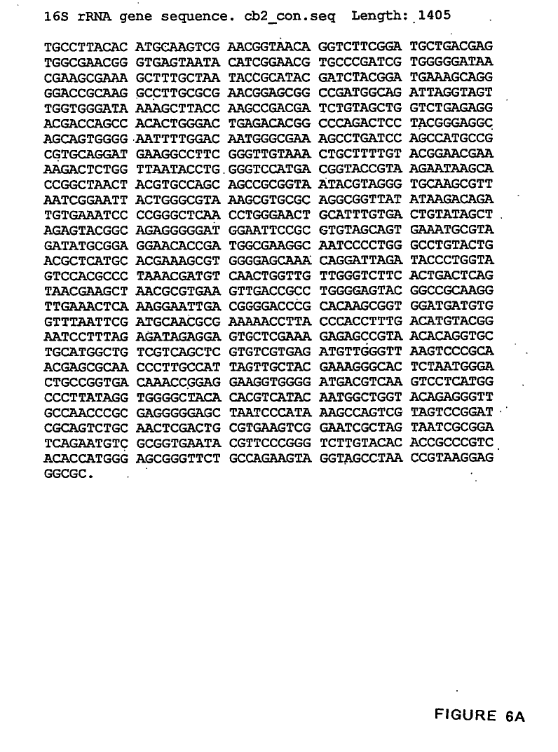

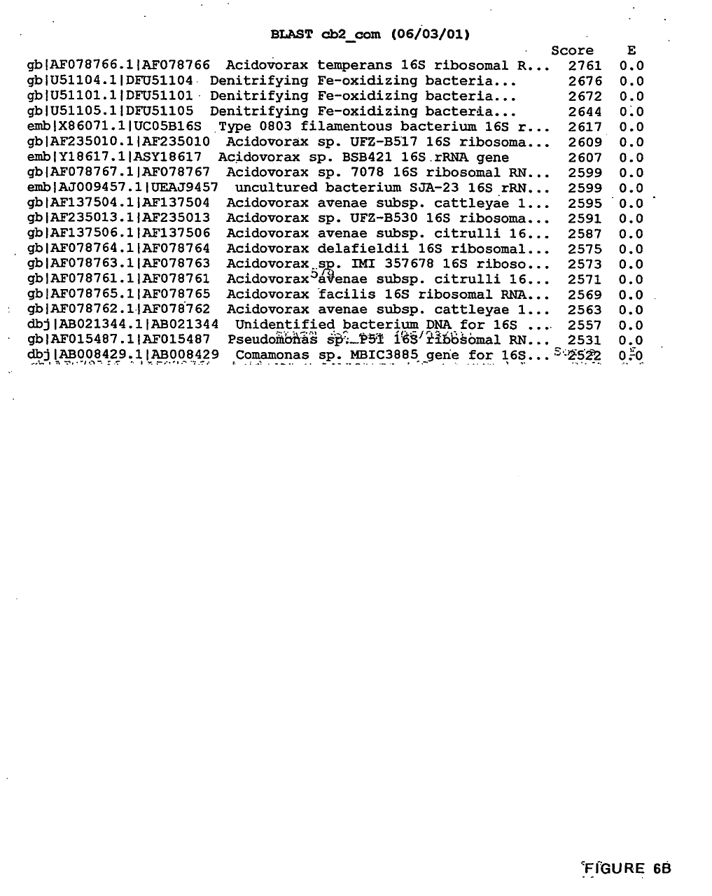

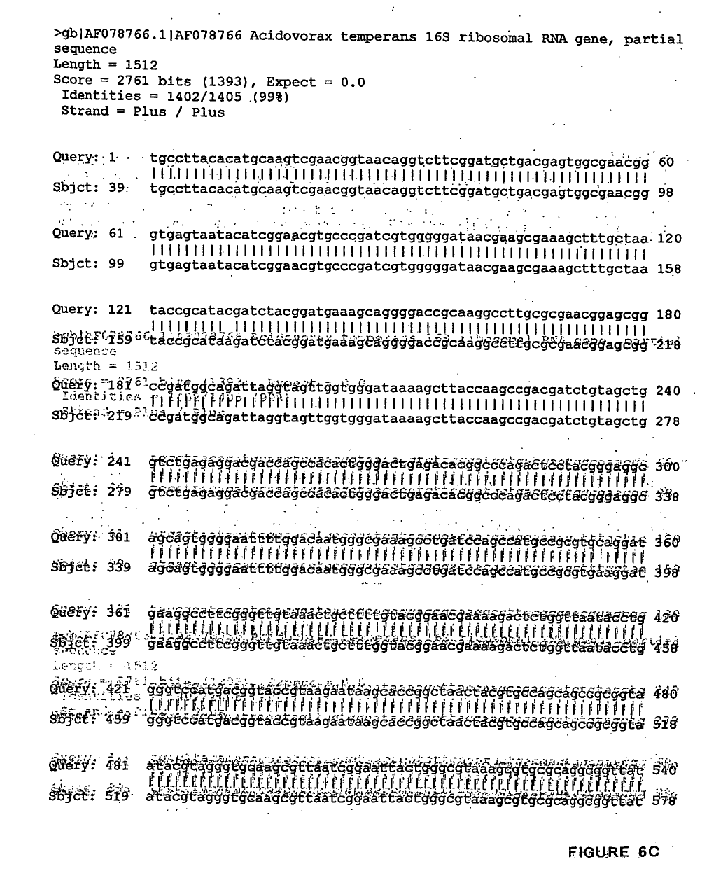

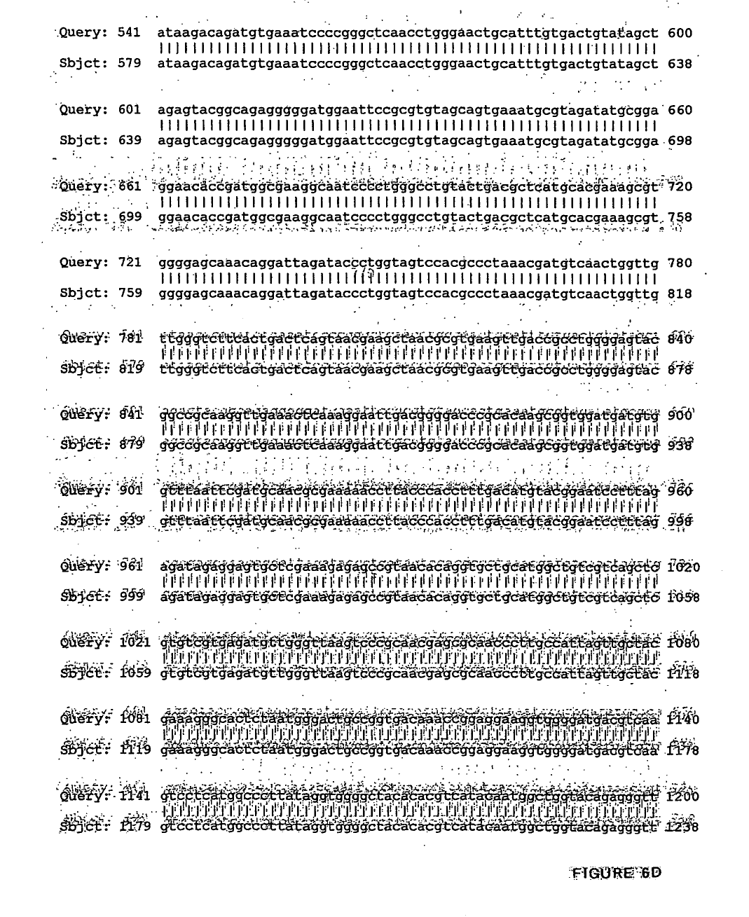

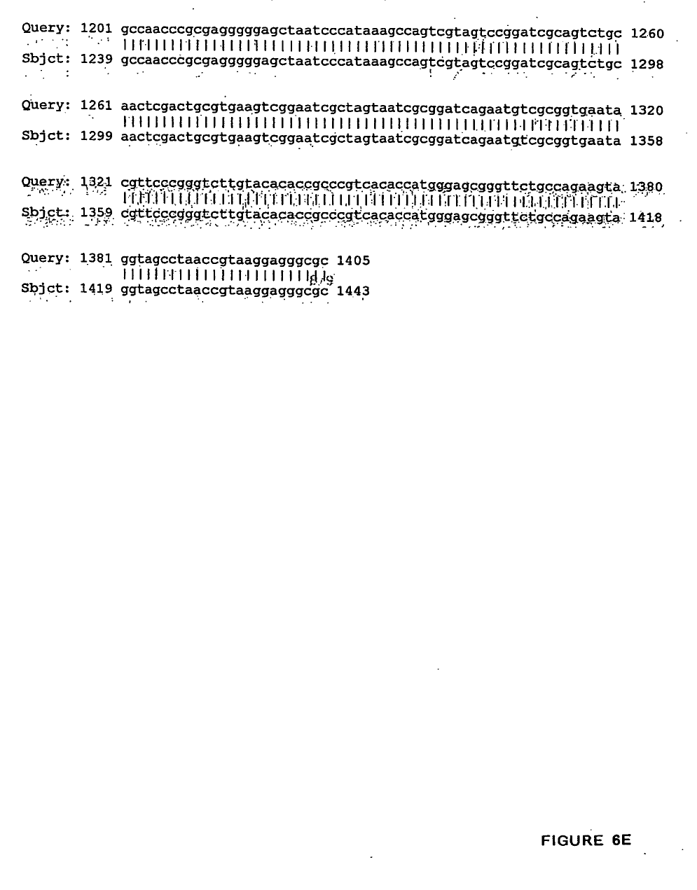

The H+ (halo-positive) strain is observed to have rough colony forms. Conversely, H− are observed to have smooth margined forms. In addition, as observed in FIG. 6, under 100× magnification the margins of H+ colonies may comprise islands of clumped cells surrounded by patches of relatively bare agar on which there are few cells. The margins of the H-colonies are smooth and comprise a consolidated margin of tightly packed bacteria.

Under dark field illumination at 100 and 400× magnification, wet mounts (in water) of H+ cells form aggregations while H− cells remain dispersed.

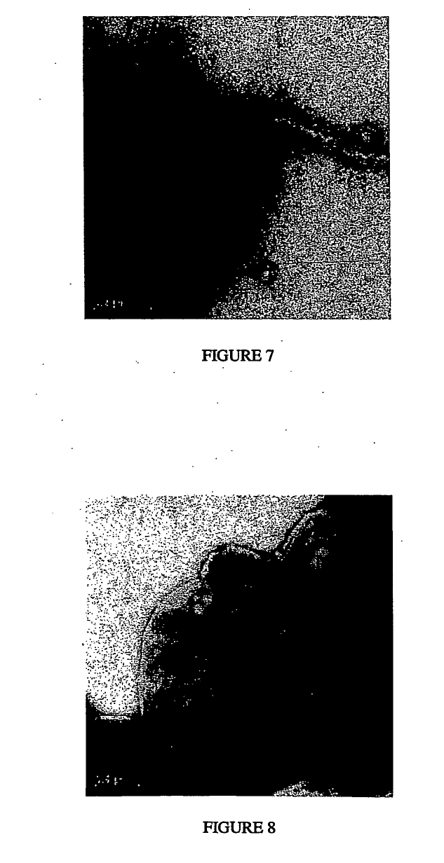

Negatively stained H+ and H− cells taken from colonies grown on R2A agar exhibit highly novel pili-like structures (referred to herein as mega-pili) on their surface, under TEM (FIG. 7, for example). The mega-pili have a large size (approximately 60-75 nm diameter×1-2 μm long), a crystalline structure and enclosed “coiled-strand”.

In addition, as observed under TEM A. temperans CB2H+ and CB2H− cells grown on R2A media have paracrystalline surface layers (FIG. 7, for example), that are reminiscent of S-layers described for a range of other bacteria (Sleytr and Beveridge, 1999).

H+ and H− are able to utilise a range of organic acids, amino acids and glycerol but do not generally utilise carbohydrates as an energy source:

The growth of both A. temperans H+ and H− may be inhibited in the presence of NaCl. More specifically, growth may be significantly inhibited by NaCl concentrations greater than approximately 0.5%. NaCl. Good growth rates may be achieved in R2A, Terrific and Nutrient media. However, comparatively poor growth is observed using both Luria broth and Luria agar.

A characteristic feature of H+ cultures is the ability to form biofilms, as demonstrated in the Examples herein after. H− cultures do not exhibit the ability to form biofilms. However, H− cultures have the ability to form flocs which settle rapidly and become tightly compressed.

16S rRNA gene sequences amplifed from a single colony isolate using universal PCR primers that amplify an approximately 1500 bp fragment corresponding to positions numbering 8-1509 of the E. coli 16S gene is provided in FIG. 6. The nucleic acid sequence obtained may be referred to herein as SEQ ID NO.5. It will be appreciated that the 16S rRNA of individual cultures of A. temperans H+ or H− may differ slightly from SEQ ID NO.1. For example, there may be micro sequence variation. Generally, such variant sequences will exhibit at least 99% sequence identity with SEQ ID NO.5.

As seen in FIG. 4, H+ and H− cells from colonies grown on R2A agar exhibit differences in protein expression. H+ expresses an approximately 40 kDa protein, as measured by SDS PAGE. H− expresses two distinct proteins of approximately 40 and 42 kDa, as measured by SDS PAGE. These proteins are characterised by novel amino acid sequence data obtained by trypsin digestion:

| | | |
|---|---|---|
| Strain | Sequence | Identification |

| | | |
|---|---|---|
| H+ | IIANDNALGAGLAFYAA | SEQ ID NO.1 |
| H+ | YPNQFQVY(S/N)PYX | SEQ ID NO.2/6 |
| H- | EVEVLXYX | SEQ ID NO.3 |
| H- | AAGLIPVAH(?)G(?)SQ | SEQ ID NO.4 |

It will be noted that standard single letter amino acid nomenclature is used herein (see “Biochemistry”, Mathews and van Holde, 1990, The Benjamin/Cummings Publishing Company Inc, California).

[S/N] above indicates the presence of either one or another amino acid at a particular position. In accordance with this the peptide YPNQFQVY(S/N)PYX has been allocated two sequence identification numbers; one includes S at position 9, and the other N.

(?) after an amino acid signifies a degree of uncertainty as to the precise identity of the immediately preceding amino acid.

‘X’ represents the presence of an amino acid of unknown identity.

On the basis of a lack of identity of these peptides with any protein sequences held within GenBank, the inventor believes novel proteins not previous identified or characterised have been discovered.

The presence of these protein sequences may be used to further characterise the novel bacterial strain of the invention. It will be appreciated that there may be some amino acid sequence variation in the above peptides between individual cultures of A. temperans H+ and those of A. temperans H−, and the invention is intended to encompass this. Such variant sequences may include amino acid substitutions, particularly conservative amino acid substitutions, for example.

Cultures of A. temperans H+ and H− may be grown to obtain larger populations or cultures by culturing with any one of a range of common bacterial media. Media containing organic acids, amino acids or peptone are particularly preferred. Examples of suitable growth media include Nutrient agar, R2A agar, and Terrific Broth (Difco Laboratories). Person's of ordinary skill in the art to which the invention relates may appreciate alternative media. While, Luria broth or Luria agar may be used in culturing the bacteria, the alternative media above are preferred.

As mentioned herein before, it is preferable that cultures are grown in media having minimal or no NaCl. Preferably conditions are such that less than approximately 1% NaCl is present in the environment in which they are grown, more preferably less than approximately 0.5% NaCl.

Generally, the conditions of culture are any which are suitable for aerobic chemo-organotrophs. However, by way of example, fresh cultures can be prepared using inocula from broth suspensions including those previously stored at −80° C. in 15% glycerol, or from soft agar stab cultures or plates stored at 28° C. Where cultures are prepared from soft agar stab cultures stored at 28° C., it is preferably that the storage time of the soft agar stabs is 2 months or less. It is preferable that cultures are agitated during incubation (for example at 100-200 rpm). To ensure optimal growth of both H+ and H− strains, incubation temperatures between approximately 28 to approximately 30° C. are preferable. Under the above exemplary conditions cultures will generally reach log growth phase within 18-24 hours.

Cultures may be harvested in accordance with standard procedures known in the art.

Cultures of the invention may be stored under any appropriate conditions for bacterial culture storage known in the art. However, in a preferred example log phase (18 hour) cultures grown in R2A broth, may be stored in 15% glycerol at −80° C.

As noted herein before, the H+ bacteria of the invention are capable of forming a biofilm and the H− bacteria have good flocculation and settling capacity. Inasmuch as this is the case, further embodiments of the invention relate to methods of optimising biofilm and flocculation capacity of compositions, as well as methods of predicting the ability of such compositions to form biofilms, flocs, and/or their capacity to settle. It will be appreciated that these methods have particular application in various industrial processes or systems, for example in bioremediation and biofiltration including waste water or effluent treatment systems.

The benefits of being able to manipulate the formation of biofilm, or flocculation and/or settling of bacteria in a composition will be readily appreciated by skilled persons. However, by way of example in the case of waste water treatment systems utilising activated sludges, the ability of bacteria to efficiently flocculate and settle aids in the separation of biomass from liquor which is subsequently released into the environment. In waste water systems which utilise biofilms which adhere to treatment plant surfaces, efficient formation of biofilms has the advantage of ensuring that a stable microbial community is maintained and that solids are not released into the effluent stream.

In one embodiment the invention provides a method of promoting the formation of a biofilm in a composition, the method including at least the step of adding to the composition Acidovorax temperans H+. The invention also provides a method of promoting flocculation of bacteria in a composition, the method including at least the step of adding to the composition Acidovorax temperans H−.

As used in relation to these embodiments of the invention the term “composition” should be taken in broad terms. It is intended to include bacterial cultures (containing a single type of bacteria, or mixed cultures of two or more different types of bacteria), other compositions containing bacteria, as well as compositions which may be absent of bacteria but which are desired to be seeded with bacteria of the invention. Examples of such compositions include activated sludge, raw sewage and biosolids. Persons of ordinary skill in the art may appreciate other such compositions.

Acidovorax temperans H+ and Acidovorax temperans H− may be added to a composition in accordance with the invention in the form of a liquid culture containing either one or both of the bacteria. Skilled persons will readily appreciate alternative forms in which the bacteria may be added to a composition. For example, freeze-dried preparations, or cell masses scraped from surface-film bioreactors, may be used.

In a particularly preferred embodiment, the culture added to the composition contains either Acidovorax temperans H+ or Acidovorax temperans H− alone.

Once added to the composition or system, it may be incubated under conditions suitable to promote the growth of either or both of Acidovorax temperans H+ and Acidovorax temperans H−. Such suitable conditions will be readily appreciated by skilled persons having regard to the information contained herein and the environment in which the composition resides (for example the operating conditions of an industrial system in which the composition is present). However, by way of example, suitable conditions include temperatures of between approximately 25° C. to approximately 28° C.

In a particularly preferred form the invention provides a method of water treatment including at least the step of adding to a water treatment system either or both of Acidovorax temperans H+ and Acidovorax temperans H−.

As used herein, the term a “water treatment system” should be taken broadly. It is intended to refer to any treatment systems or processes known in the art, for example activated sludge processes, fixed film bioreactors and batch reactors. In addition, reference to a water treatment system or process should not be taken as a reference to the addition of bacteria at a particular time in the process or to a particular physical part of a treatment plant. In activated sludge processes it is preferable that the bacteria of the invention are added to the activated sludge within the system, however it will be appreciated that they may also be added elsewhere. For example, bacteria of the invention may be used to seed raw wastewater influent and influent to sludge digesters.

In addition, it will be appreciated that at one particular time during an industrial process it may be desirable to promote biofilm formation. At a distinct time in the process, it may be desirable to promote flocculation; for example where it is desired to separate biomass from liquor in a water treatment process. This may be readily achieved in accordance with the invention by adding the bacteria to the system at appropriate times.

It will be appreciated that in the methods of the invention, where flocculation of bacteria is desired, Acidovorax temperans H− is added, and where formation of biofilm is desired, Acidovorax temperans H+ is added.

In addition to the above methods, the invention also provides a method of predicting the likelihood, or ability, of bacteria in a composition to flocculate, or to form a biofilm, the method including at least the step of detecting the presence of Acidovorax temperans H− and/or Acidovorax temperans H+. In a preferred embodiment, the method involves detecting the presence of both Acidovorax temperans H+ and Acidovorax temperans H−. More preferably, the ratio of Acidovorax temperans H+ to Acidovorax temperans H− is determined. A high ratio of Acidovorax temperans H+ to Acidovorax temperans H− may be predictive of the likelihood biofilm formation. A low ratio of Acidovorax temperans H+ to Acidovorax temperans H− may be predictive of the likely ability to flocculate and/or settle. By way of example, the inventor contemplates the ratio of one bacteria to the other differing by a log order of magnitude may be so predictive.

Detecting the presence of bacteria of the invention may be effected using standard microbiological techniques, having regard to the specific information contained herein. By way of example, bacteria may be isolated from a composition by spread plate techniques on R2A media. Presumptive colonies may then be identified by colony morphology, and cell morphology and re-streaked on fresh media to obtain pure cultures. The presence of bacteria of the invention may then be determined on the basis of one or more of the distinctive characteristics described herein. For example, the bacterial cultures may be visualised under the light microscope to observe their characteristic colony morphology, individual bacteria may be observed under TEM to observe their characteristic cellular morphology, SDS PAGE of isolates of cell surface proteins may be conducted, as well as sequencing of tryptic peptides as herein beforementioned. In addition, characteristic flocculation capacities and the like may be analysed. Other means of determining the presence of the bacteria of the invention may be apparent from material elsewhere herein.

As should be appreciated, once the presence of bacteria of the invention has been determined as mentioned above, this information may be used to manipulate conditions in a composition or system to increase the likelihood of bacteria present to form biofilms or their capacity to flocculate and/or settle. Where flocculation is desirable one may add Acidovorax temperans H− to encourage or promote this action. Where biofilm formation is desired, Acidovorax temperans H+ may be added to aid this.

In accordance with this, methods of the invention may further include the step of detecting the presence of one or both of Acidovorax temperans H+ and Acidovorax temperans H− in a composition, prior to adding one or both of Acidovorax temperans H+ and Acidovorax temperans H− thereto. The choice of bacteria added will be dependent on the results obtained and whether or not flocculation or formation of biofilm has been predicted, or is desired.

In an additional embodiment of the invention there is a method of maintaining viability of, and/or promoting growth of, Acidovorax temperans H+ and/or Acidovorax temperans H− in a composition, including at least the steps of determining the NaCl concentration in the composition, and where the concentration is above approximately 1%, reducing it to below approximately 1%. More preferably, the NaCl concentration is reduced to below approximately 0.5%.

It will be appreciated that this aspect of the invention is applicable to the situation where Acidovorax temperans H+ and/or H− are in an industrial system, such as in activated sludge in a waste water treatment system.

NaCl concentration may be determined by any techniques standard in the art to which the invention relates. By way of example, NaCl concentration may be measured using a salinity meter or indirectly assessed through the measurement of conductivity.

The NaCl concentration in a composition in which the Acidovorax temperans H+ and/or H− are present, may be reduced by any number of means known in the art. However, by way of example, the composition may be diluted using water, fresh wastewater, effluent, or other appropriate liquid.

As will be appreciated from the information herein before, the identification of two novel strains of Acidovorax temperans and their use in industrial systems, for example water treatment processes, to promote floc or biofilm formation, has significant commercial application.

In particular, the use of the distinctive H+ and H− colony phenotypes for the prediction and detection of biofilm and floc formation in industrial processes generally, allows the development of process optimisation based on the detection of the either or both novel strains of Acidovorax temperans and manipulation of their ratios. For example, the use of the two phenotypes could be manipulated as described herein before for the promotion of floc formation in industrial processes in general (and waste water treatment in particular), or the promotion of biofilm formation in treatment processes utilising fixed growth reactors.

It will be appreciated that the invention is applicable to existing processes, systems and industrial plants as will be readily recognised by skilled persons.

### EXAMPLES

Isolation of Acidovorax from Activated Sludge

Bacterial strain CB2, was isolated in pure culture from activated sludge on R2A agar (Difco Labroatories, Maryland, USA) incubated at 28 C for 5 days. Isolates were grown to late-log phase in R2 broth and were stored at −70° C. Freezer stocks were supplemented with 15% glycerol as a cryoprotectant.

Colonies from the original isolation were described as small (1.5-2.5 mm diameter), creamy yellow coloured, raised, and having an entire margin. Cells were observed under light microscopy to be Gram negative rods measuring approx. 0.5 μm×1.5-2.0 μm. Sudan Black staining indicated the intracellular presence of Poly B-hydroxybutyrate.

Biochemical capability and substrate utilisation patterns were determined for cultures grown on R2A agar using BBL Crystal Enteric/NF and BIOLOG GN2 minaturised assays. Both assays were performed as generally described in the manufacturers instructions, with the exception that test plates were incubated at 28° C. for up to 5 days. CB2 generally yielded negative results for carbohydrates, but was able to utilise a range of organic acids, amino acids and glycerol.

Comparative DNA sequence analysis of the 16S rRNA gene sequences that were PCR amplifed from a single colony isolate showed 99% sequence homology over a 1405-bp region to Acidovorax temperans strain CCUG 11779, GenBank Accession # AF078766 (FIG. 6).

A. temperans groups within the beta Proteobacteria. The genus Acidovorax was defined by Willems et al (Int. J. Syst. Bacteriol. 40: 384-398) and is described in Bergey's Manual of Bacteriology (Holt et al Eds, 9th Edition) as comprising Gram negative, oxidase positive, straight to slightly curved rods. Good growth is obtained on media containing organic acids, amino acids or peptone, but only limited number of sugars are used for growth. A. temperans is also reported to be capable of heterotrophic denitrification of nitrate.

These characteristics are consistent with the morphological and biochemical data obtained for CB2. However, as noted elsewhere herein the novel organisms of the invention exhibit certain distinguishing characteristics.

Identification of Two Colony Phenotypes of Acidovorax

Freezer stocks of CB2 were used to inoculate R2A agar plates, which were then incubated in an ambient temperature waterbath (average temperature of 24° C.).

Surprisingly, following incubation for seven days, two distinct colony morphologies were observed. One group had a rough margin, giving a distinct halo-like appearance while the other group had regular “entire” margins. Rough colony forms were subsequently referred to as “halo-positive” (H+). Conversely the smooth margined forms were referred to as “halo-negative” (H−). Colony descriptions in the literature for members of the genus Acidovorax describe the colonies as having entire margins. The rough H+ variant would therefore appear to be a new observation for this organism.

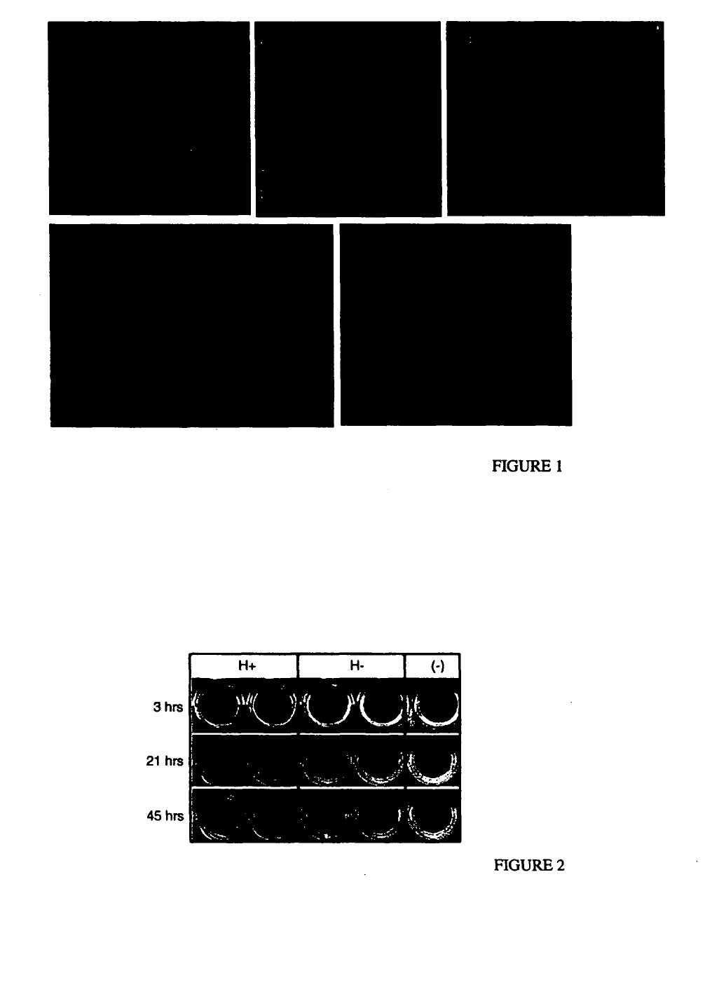

Examination of the two colony types under 100× magnification revealed distinct differences in the dispersion of individual cells over the agar surface (FIG. 1). The margins of H+ colonies comprised islands of clumped cells surrounded by patches of relatively bare agar on which there were few cells. In contrast the margins of the H− colonies were smooth and comprised a consolidated margin of tightly packed bacteria.

To examine the cells more closely wet mounts of cell mass were prepared in water from H+ and H− colonies and viewed under dark field illumination at 100 and 400× magnification. Cells from H+ colonies formed aggregations while those from H− colonies remained dispersed.

Factors influencing the development stability of the two phenotypes have been investigated by multiple subculturing of H+ and H− colonies on R2A agar and also in R2 broth. Both phenotypes are relatively stable although there is some evidence of switching, albeit infrequently between types.

The H+ phenotype develops only after prolonged incubation (3-5 days) and during the 1-3 days of culture, the colonies have entire (H−) margins. The development of the H+ phenotype is suppressed for a further period when cultures are grown on protein-rich media Based on these observations it is proposed that expression of the H+ phenotype is influenced by nutritional cues and/or some other cell density-related signal.

Demonstration That Phenotype H+ Forms Biofilms Whereas H− Does Not

The capacity of the H+ and H− phenotypes to attach to surfaces and initiate biofilm formation was investigated using a crystal-violet staining method as described by Kjaergaard et al (2000). Biofilms were observed after incubation for 3, 21 and 45 hours. Cultures derived from H+ colonies formed biofilms within 3 hours of inoculation into fresh R2 media while H− cells required incubation for between 21 and 45 hours before significant cell attachment was observed (FIG. 2). Biofilms formed by H− after such long incubation times were less stable than H+ and could be removed with vigorous washing. H+ notably formed very strong biofilms which were not disturbed by vigorous washing. On this basis it is concluded that the rough (H+) phenotype is indicative of biofilm forming capacity.

The H+ and H− Forms Differ in their Ability to Flocculate

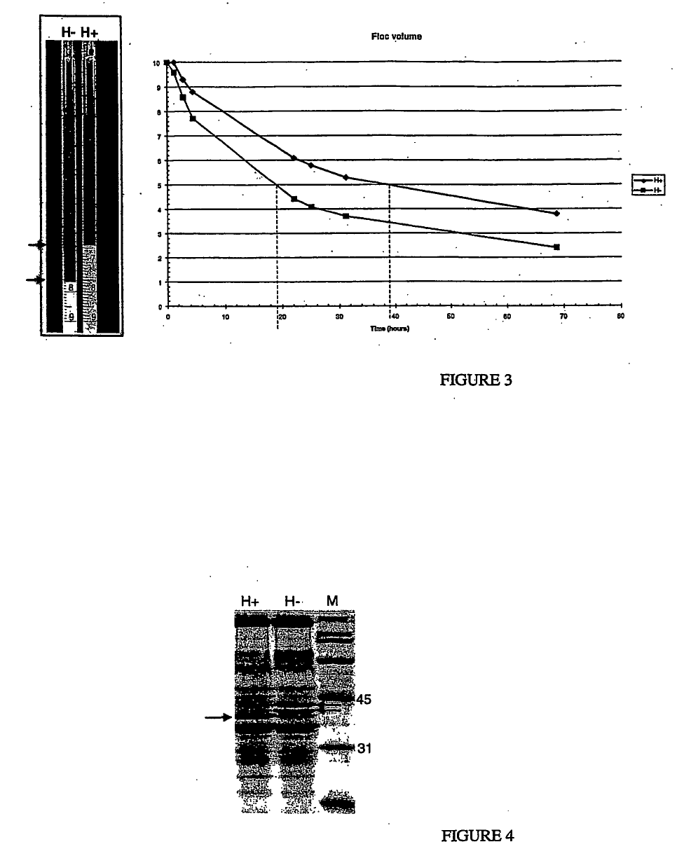

The ability of the H+ and H− phenotypes to flocculate and settle in broth culture was investigated by inoculating 30 mls of nutrient-rich Terrific Broth with a single colony isolate representing each colony morph. Cultures were incubated on a shaking platform at 28° C. for 3 days yielding turbid stationary-phase cultures. Both H+ and H− appeared to produce large amounts of extracellular polymers and were viscous. The settlement rate of flocs was determined by drawing 10 mls of cell suspension into a 10 ml graduated pipette, which was capped and allowed to stand at ambient temperature. The relative proportions of cleared supernatant and settled floc were recorded over a 31 hour period. The results (FIG. 3) indicate that H− flocs settle rapidly forming a tightly compressed pellet while H+ cells settle more slowly and formed a less compressed floc. On this basis it is concluded that the smooth (H−) phenotype is indicative of good flocculation and settling capacity.

Discovery that the Surface Proteins on the Cells Differ for H+ and H− Forms

Differences in protein expression between H+ and H− colony types were investigated using 1D SDS PAGE and 2D proteome methods. Cell surface-associated proteins, were prepared for analysis by heating cell suspensions to 60° C. for 10 minutes and precipitating the proteins from the supernatants. SDS PAGE analysis of supernatant fractions revealed three major differences in protein expression between H+ and H− phenotypes (FIG. 4).

Extracts from H+ colony preparations yielded an approximately 40 kDa protein that is up-regulated compared with that of H− cells. In contrast extracts from H+ yielded two significantly up-regulated protein bands (approx 40 and 42 kDa).

Discovery that the Surface Proteins are New and Able to be Identified by their Partial Amino Acid Sequence

Amino acid sequencing was performed on two trypsin digest fractions from each of the 40 kDa H+ surface-associated protein and the larger (42 kDa) H− surface-associated protein. The following sequences were obtained:—

| | |
|---|---|
| H+ Fraction 16 | (IIANDNALGAGLAFYAA) |
| H+ Fraction 18 | (YPNQFQVY(SIN)PYX). |
| H- Fraction 18 | (EVEVLXYX) |
| H- Fraction 29 | (AAGLIPVAH(?)G(?)SQ) |

Neither sequence showed identity to currently described protein sequences within Genbank database.

Identification of the Surface Structures that Correlate with Biofilm and Floc Formation

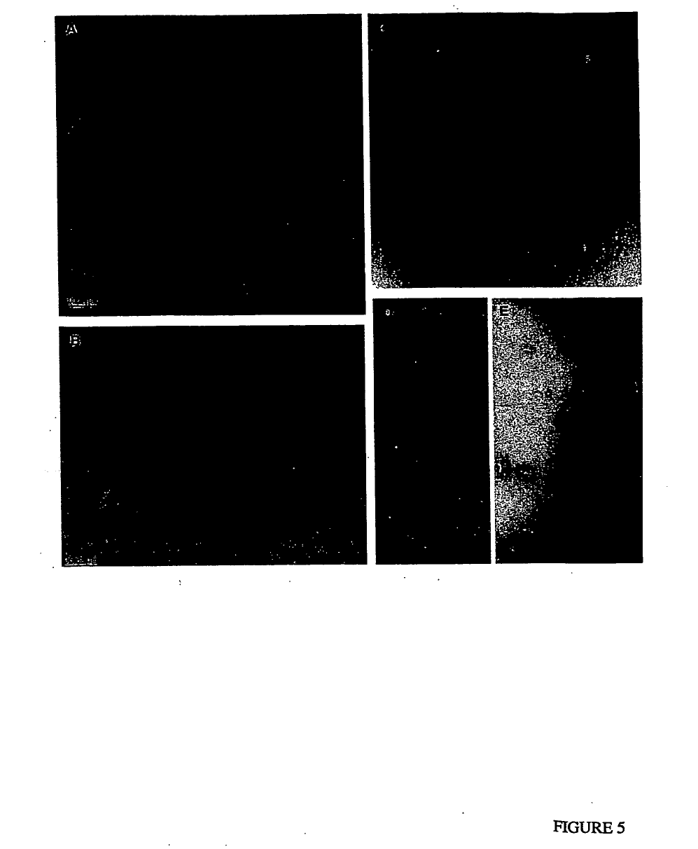

The cell surface morphology of H+ and H− phenotypes was investigated by Transmission Electron Microscopy of negatively stained colony suspensions prepared using 1% phosphotungstic acid. This initial analysis revealed different surface structures associated with the two phenotypes (FIG. 5). H+ cells are surrounded by long, thin (0.02 μm) fibrils that resemble pili described for other Gram negative organisms. H+ cells also appeared to be surrounded by a matrix of extracellular material. H− cells were covered in thick (approx 0.2 μm) appendages that appeared flexible and which also appear to mediate attachment between cells. These appendages appear as tube-like structures with internal flexible strand.

The observation of different surface structures correlates with the different protein bands, described above and the observed differences in biofilm and floc settlement recorded for H+ and H− colony types.

Both CB2H+ and CB2Hneg Produce Novel Mega-Pili

Following the above experiments, the inventor conducted further TEM studies of negatively stained A. temperans CB2H+ and CB2H− cells grown on R2A media. These studies revealed the presence of highly novel pili-like structures, that the inventor refers to as mega-pili, on the surface of both CB2H+ and CB2Hneg strains. These mega-pili are seen in FIG. 7. Detailed TEM studies indicate that the structures are consistently produced by both strains. TEM analysis carried out on the related species Acidovorax delafieldii, grown under identical conditions, revealed that these cells are flagellated, but do not produce megapili.

The large size (60-75 nm diameter×1-2 μm long), crystalline structure and enclosed “coiled-strand” of the mega-pili renders these structures totally unique and unlike any other bacterial cell-surface appendage described to-date. Previous descriptions of the physiology and morphology of Acidovorax temperans make no reference to these structures, although previously identified cells have been described as having polar flagella Neither CB2H+ or CB2Hneg produce flagella when grown on R2A media. The inventor believes the megapili are unique to the strains identified herein.

Both CB2H+ and CB2Hneg Produce Paracrystalline Surface Layers (S-Layers)

TEM analysis of negatively stained A. temperans CB2H+ and CB2H− cells grown on R2A media revealed the presence of paracrystalline surface layers (FIG. 7) that are reminiscent of S-layers described for a range of other bacteria.

S-layer proteins have been studied in a wide variety of bacteria, and although they share structural features, the amino acid composition is highly variable and genetic sequences show little homology (Bahl et al, 1997). On this basis, the inventor believes that the S-layers found on A. temperans are unique to this species.

The Growth of A. temperans CB2 is Inhibited in the Presence of NaCl.

During the course of the inventors work attempts were made to grow CB2 on a variety of complex microbiological media including R2A, Nutrient Agar/broth, Terrific Broth, and Luria agar/broth. While good growth rates were achieved in R2A, Terrific and Nutrient media, comparatively poor growth was observed using both Luria broth and Luria agar. These four media differ in the nature and concentrations of organic nutrients and the presence of salts. Luria broth/agar (1% tryptone, 0.5% yeast extract 1% NaCl) is the only media containing NaCl raising the question of whether the growth of CB2 was inhibited by the presence of NaCl.

To investigate the effect of NaCl on the growth of CB2, Nutrient agar (0.5% peptone, 0.3% beef extract, 1.5% agar) plates was prepared containing varying NaCl concentrations. Individual plates were streaked for single colonies with A. temperans CB2H+, A. temperans CB2Hneg B subtilis, E. coli and A. delafieldii, incubated for 48 hours at 28 C and observed for growth. Growth on NaCl supplemented plates was scored in terms of colony size relative to that on plates containing no NaCl. The results are seen in Table 1 below.

TABLE 1
Effect of NaCl concentration on growth
of bacteria on nutrient agar

| | A. temperans | A. temperans | A. | E. | B. |
|---|---|---|---|---|---|
| | CB2 H+ | CB2 Hneg | delafieldii | coli | subtilis |
| NB | +++ | +++ | +++ | +++ | +++ |
| NB + 0.5% | +++ | +++ | +++ | +++ | +++ |
| NaCl | | | | | |
| NB + 1.0% | ++ | ++ | ++ | +++ | +++ |
| NaCl | | | | | |
| NB + 1.5% | +/− | +/− | +/− | ++ | +++ |
| NaCl | | | | | |

NB, nutrient broth. Growth is scored as colony size relative to growth in the absence of NaCl (rated as +++). Growth scored as +/− was barely visible and individual colonies could not be discerned.

The results above indicate that the growth of both A. temperans and A. delafieldii is significantly inhibited by NaCl at concentrations greater than 0.5%. NaCl concentrations may vary in wastewater depending on the degree of seawater intrusion to the system and the proportion of high-salt domestic and/or industrial wastewater in the influent.

The results of the present work indicates that NaCl levels exceeding 0.5% are inhibitory to A. temperans and therefore the inventor proposes that:—
(i) NaCl concentrations in the influent and mixed liquor may be used as a predictor of optimal conditions for growth of A. temperans in activated sludge
(ii) NaCl measurements may be used as predictor of conditions deleterious to A. temperans performance
(iii) manipulation of NaCl concentrations in the wastewater treatment process may be used to ensure optimal conditions for A. temperans growth and as a consequence optimal treatment performance.

### Working Example 1

### Waste Water Treatment

Activated sludge flocculation ability and hence solids removal from effluent may be improved through the manipulation of the component Acidovorax temperans H+ and H− populations. This example demonstrates how the enhanced flocculation capability of H− strains may be utilised to generally enhance the flocculation of activated sludge bacterial community through entrapment of other bacteria/solids within the floc.

A sample is taken from activated sludge within a treatment system. Bacteria present within the sample are isolated by spreading on R2A media plates. H− and H+ colonies are identified by morphology, and the relative number of colonies of H+ and H− observed. Confirmation of identity of the colonies is completed on the basis of their distinct characteristics using one or a combination of light microscopy, TEM, SDS PAGE of isolates of cell surface proteins, sequencing of tryptic peptides of unique surface proteins, or characteristic flocculation capacities, in accordance with the techniques described herein before.

In the absence of H−, or where there is a low proportion of H− compared to H+, an enriched log phase liquid culture of H− is added to the return activated sludge by drip feed. Temperature within the activated sludge is monitored to maintain it within the range 25 to 28° C. to promote growth of Acidovorax temperans H−.

Following addition of the H− culture, flocculation characteristics of the sludge are monitored by observation of the biomass. The growth of the H− population relative to H+, or to other bacterial species, may also be monitored according to the techniques described above. Biomass may be separated from liquor according to standard procedures, for example by gravity settlement or desolved air floatation (DAF). Liquor is then returned to the environment.

### Working Example 2

The flocculation ability of biomass within activated sludge of a water treatment system is observed. Where flocculation is minimal, or it is observed that flocs are weak or bouant and have difficulty settling, an enriched log phase culture of H− is added by drip feed to the raw sewage inflow.

The system is then maintained and monitored as for Working Example 1. Biomass may be separated from liquor according to standard procedures. Liquor is then returned to the environment.

### Working Example 3

Protocols for determining the presence of H+ and/or H− bacteria, and for adding H− to a water treatment system, are as described in Working Example 1. However, the process includes an additional step of monitoring the levels of NaCl in the treatment system using a salinity meter. Where NaCl levels are above approximately 0.5% sufficient effluent is added to drop the concentration to below approximately 0.5%.

Biomass is separated from liquor according to standard procedures. Liquor is then returned to the environment.

The above working examples outline processes that may be particularly beneficial for systems suffering from problems with “weak” or excessively buoyant flocs.

While in the foregoing description there has been made reference to specific components or integers of the invention having known equivalents then such equivalents are herein incorporated as if individually set forth.

Although this invention has been described by way of example only and with reference to possible embodiments thereof it is to be understood that modifications or improvements may be made without departing from the scope of the invention. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in New Zealand, or any other country.

### REFERENCES

Davey, M and O'Toole, G. 2000 Microbial biofilms: from ecology to molecular genetics. Microbiology and Molecular Biology Reviews 64(4) 847-867.
Willems, A., Falsen, E., Pot, B., Jantzen, E., Hoste, B, Vandamme, P., Gillis, M., Kersters, K., De-Ley. J. 1990. Acidovorax, a new genus for Pseudomonas facilis, Pseudomonas delafeldii, E. Falsten (EF) group 13, EF group 16, and several clinical isolates, with the species Acidovorax facilis comb. nov., Acidovorax delafieldii comb. nov., and Acidovorax temperans sp. nov. Internatioanl Journal of Systematic Bacteriology. 40: 384-398.
Kjaergaard et al 2000 Journal of Bacteriology 182(17) 4789-4796.
Brosius, J., Dull, T. L., Sleeter, D. D. and Noller, H. F. (1981). Gene organisation and primary structure of a ribosomal RNA operon from Escherichia coli. Journal of Molecular Biology. 148, 107-127
Sleytr U B & Beveridge T J (1999) Bacterial S-layers. Trends in Microbiology 7(6):253-260
Bahl H et al FEMS Microbiology Reviews 20 (1997) 47-98

1-27. (canceled)

28. An isolated bacterium, Acidovorax temperans H+, said bacteria having the following characteristics: gram negative; substantially rod-shaped cells; colonies of a substantially rough form; capable of growth in/on R2A, R2, nutrient and/or Terrific media at 28° C.; intracellular presence of Poly β-hydroxybutyrate as observed using Sudan Black staining of cells grown in Terrific broth with aeration; Mega-pili on the surface as observed by TEM in cells grown on R2A agar; paracrystalline surface layers as observed by TEM in cells grown on R2A agar; a 16S rRNA sequence of SEQ ID NO.5, or variant thereof; and capable of forming biofilms as observed using crystal-violet staining of cells grown in R2 broth.

29. Acidovorax temperans H+ as claimed in claim 28 wherein the mega-pili are substantially 60-75 nm in diameter and substantially 1-2 μm long, having a substantially crystalline structure and enclosed coiled-strand.

30. Acidovorax temperans H+ as claimed in claim 28 which is sensitive to growth in NaCl.

31. Acidovorax temperans H+ as claimed in claim 28 further characterised by the expression of an approximately 40 k Da protein, as measured by SDS PAGE, following growth on R2A agar.

32. Acidovorax temperans H+ as claimed in claim 31 wherein the protein is characterised by the presence of one or more peptide of SEQ ID NO.1, 2 and 6.

33. Acidovorax temperans H+characterised by the deposit NM03/35786.

34. An isolated bacterium, Acidovorax temperans H−, said bacteria having the following characteristics: gram negative; substantially rod-shaped cells; colonies having a substantially smooth margin; capable of growth in/on R2A, R2, nutrient and/or Terrific media at 28° C.; intracellular presence of Poly B-hydroxybutyrate as observed using Sudan Black staining of cells grown in Terrific broth with aeration; Mega-pili on the surface as observed by TEM in cells grown on R2A agar; paracrystalline surface layers as observed by TEM in cells grown on R2A agar; a 16S rRNA sequence of SEQ ID NO.5, or variant thereof; and capable of flocculation when grown in nutrient rich Terrific broth.

35. Acidovorax temperans H− as claimed in claim 34 wherein the mega-pili are substantially 60-75 nm in diameter and substantially 1-2 μm long, having a substantially crystalline structure and enclosed coiled-strand.

36. Acidovorax temperans H− as claimed in claim 34 which is sensitive to growth in NaCl.

37. Acidovorax temperans H− as claimed in claim 34 further characterised by the expression of an approximately 40 kDa protein and 42 kDa protein, as measured by SDS PAGE, following growth on R2A agar.

38. Acidovorax temperans H− as claimed in claim 37 wherein the proteins are characterised by the presence of one or more peptide of SEQ ID NO.3, and 4.

39. Acidovorax temperans H− characterised by the deposit NM03/35787.

40. A culture of Acidovorax temperans H+ of claim 28.

41. A culture of Acidovorax temperans H+ of claim 34.

42. A method of promoting the formation of a biofilm in a composition, the method including at least the step of adding to the composition Acidovorax temperans H+ as claimed in claim 28.

43. A method of promoting flocculation of bacteria in a composition, the method including at least the step of adding to the composition Acidovorax temperans H− as claimed in claim 34.

44. A method as claimed in claim 42 wherein the composition is a bacterial culture.

45. A method as claimed in claim 43 wherein the composition is a bacterial culture.

46. A method as claimed in claims 42 wherein the composition is activated sludge or sewage.

47. A method as claimed in claims 43 wherein the composition is activated sludge or sewage.

48. A method of water treatment, the method including at least the step of addling to a water treatment system either or both of isolated bacteria Acidovorax temperans H+ and Acidovorax temperans H−, wherein the Acidovorax temperans H+ has the following charateristics: gram negative; substantially rod-shaped cells; colonies of a substantially rough form; capable of growth in/on R2A, R2, nutrient and/or Terrific media at 28° C.; intracellular presence of poly β-hydroxybutyrate as observed using Sudan Black staining of cells grown in Terrific broth with aeration; Mega-pili on the surface as observed by TEM in cells grown on R2A agar; Paracrystalline surface layers as observed by TEM in cells grown on R2A agar; a 16S rRNA sequence of SEQ ID NO.5, or variant thereof; and capable of forming biofilms as observed using crystal-violet staining of cells grown in R2 broth; and the Acidovorax temperans H− has the following characteristics: Gram negative; substantially rod-shaped cells; colonies having a substantially smooth margin; capable of growth in/on R2A, R2, nutrient and/or Terrific media at 28° C.; intracellular presence of poly B-hydroxybutyrate as observed using Sudan Black staining of cells grown in Terrific broth with aeration; Mega-pili on the surface as observed by TEM in cells grown on R2A agar; paracrystalline surface layers as observed by TEM in cells grown on R2A agar; a 16S rRNA sequence of SEQ ID NO.5, or variant thereof; and capable of flocculation when grown in nutrient rich Terrific broth.

49. A method as claimed in claim 48, wherein Acidovorax temperans H− is added where the promotion of flocculation of bacteria is desired.

50. A method as claimed in claim 48, wherein Acidovorax temperans H+ is added where promotion of biofilm formation is desired.

51. A method as claimed in claim 48 wherein Acidovorax temperans H+ and/or H− are added to activated sludge within the water treatment system.

52. A method as claimed claim 48 wherein, Acidovorax temperans H+ and/or Acidovorax temperans H− are added as liquid cultures.

53. A method of water treatment, the method including at least the steps of: (a) detecting the presence of one or both of Acidovorax temperans H+ and Acidovorax temperans H− in a water treatment system; and dependent on the results, (b) adding to the treatment plant an amount of either or both of Acidovorax temperans H+ and Acidovorax temperans H−, wherein the Acidovorax temperans H+ has the following charateristics: gram negative; substantially rod-shaped cells; colonies of a substantially rough form; capable of growth in/on R2A, R2, nutrient and/or Terrific media at 28° C.; intracellular presence of poly β-hydroxybutyrate as observed using Sudan Black staining of cells grown in Terrific broth with aeration; Mega-pili on the surface as observed by TEM in cells grown on R2A agar; paracrystalline surface layers as observed by TEM in cells grown on R2A agar; a 16S rRNA sequence of SEQ ID NO.5, or variant thereof; and capable of forming biofilms as observed using crystal-violet staining of cells grown in R2 broth; and the Acidovorax temperans H− has the following characteristics: gram negative; substantially rod-shaped cells; colonies having a substantially smooth margin; capable of growth in/on R2A, R2, nutrient and/or Terrific media at 28° C.; intracellular presence of poly β-hydroxybutyrate as observed using Sudan Black staining of cells grown in Terrific broth with aeration; Mega-pili on the surface as observed by TEM in cells grown on R2A agar; Paracrystalline surface layers as observed by TEM in cells grown on R2A agar; a 16S rRNA sequence of SEQ ID NO.5, or variant thereof; and capable of flocculation when grown in nutrient rich Terrific broth.

54. A method as claimed in claim 53 wherein the method further includes the step of determining the ratio of Acidovorax temperans H+ to Acidovorax temperans H− present in the treatment system.

55. A method as claimed in claim 53 wherein Acidovorax temperans H− is added where promotion of flocculation of bacteria is desired.

56. A method as claimed in claim 53 wherein Acidovorax temperans H+ is added where promotion of biofilm formation is desired.

57. A method of predicting the likelihood of bacteria in a composition to flocculate the method including at least the step of detecting the presence of Acidovorax temperans H− and/or Acidovorax temperans H+, wherein the Acidovorax temperans H− has the following characteristics: gram negative substantially rod-shaped cells; colonies having a substantially smooth margin; capable of growth in/on R2A, R2, nutrient and/or Terrific media at 28° C.; intracellular presence of poly β-hydroxybutyrate as observed using Sudan Black staining of cells grown in Terrific broth with aeration; Mega-pili on the surface as observed by TEM in cells grown on R2A agar; Paracrystalline surface layers as observed by TEM in cells grown on R2A agar; a 16S rRNA sequence of SEQ ID NO.5, or variant thereof; and capable of flocculation when grown in nutrient rich Terrific broth; and the Acidovorax temperans H+ has the following charateristics: gram negative; substantially rod-shaped cells; colonies of a substantially rough form; capable of growth in/on R2A, R2, nutrient and/or Terrific media at 28° C.; Intracellular presence of poly β-hydroxybutyrate as observed using Sudan Black staining of cells grown in Terrific broth with aeration; Mega-pili on the surface as observed by TEM in cells grown on R2A agar; paracrystalline surface layers as observed by TEM in cells grown on R2A agar; a 16S rRNA sequence of SEQ ID NO.5, or variant thereof; and capable of forming biofilms as observed using crystal-violet staining of cells grown in R2 broth.

58. A method of predicting the likelihood of bacteria in a composition to form a biofilm, the method including at least the step of detecting the presence of Acidovorax temperans H− and/or Acidovorax temperans H+, wherein the Acidovorax temperans H− has the following characteristics: gram negative substantially rod-shaped cells; colonies having a substantially smooth margin; capable of growth in/on R2A, R2, nutrient and/or Terrific media at 28° C.; intracellular presence of poly β-hydroxybutyrate as observed using Sudan Black staining of cells grown in Terrific broth with aeration; Mega-pili on the surface as observed by TEM in cells grown on R2A agar; paracrystalline surface layers as observed by TEM in cells grown on R2A agar; a 16S rRNA sequence of SEQ ID NO.5, or variant thereof; and capable of flocculation when grown in nutrient rich Terrific broth; and the Acidovorax temperans H+ has the following charateristics: gram negative; substantially rod-shaped cells; colonies of a substantially rough form; capable of growth in/on R2A, R2, nutrient and/or Terrific media at 28° C.; intracellular presence of poly β-hydroxybutyrate as observed using Sudan Black staining of cells grown in Terrific broth with aeration; Mega-pili on the surface as observed by TEM in cells grown on R2A agar; paracrystalline surface layers as observed by TEM in cells grown on R2A agar; a 16S rRNA sequence of SEQ ID NO.5, or variant thereof; and capable of forming biofilms as observed using crystal-violet staining of cells grown in R2 broth.

59. A method as claimed in claims 57 wherein the method involves detecting the presence of both Acidovorax temperans H+ and Acidovorax temperans H−.

60. A method as claimed in claims 58 wherein the method involves detecting the presence of both Acidovorax temperans H+ and Acidovorax temperans H−.

61. A method as claimed in claim 59 wherein the ratio of Acidovorax temperans H+ to Acidovorax temperans H− is determined, a high proportion of Acidovorax temperans H+ to Acidovorax temperans H− being predictive of the likelihood of biofilm formation and a high proportion of Acidovorax temperans H− to Acidovorax temperans H+ being predictive of the likelihood of flocculation.

62. A method as claimed in claim 60 wherein the ratio of Acidovorax temperans H+ to Acidovorax temperans H− is determined, a high proportion of Acidovorax temperans H+ to Acidovorax temperans H− being predictive of the likelihood of biofilm formation and a high proportion of Acidovorax temperans H− to Acidovorax temperans H+ being predictive of the likelihood of flocculation.

63. A method as claimed in claim 61 wherein a proportion of Acidovorax temperans H+ to Acidovorax temperans H− differing by a log order of magnitude is predictive of the likelihood of biofilm formation.

64. A method as claimed in claim 62 wherein a proportion of Acidovorax temperans H+ to Acidovorax temperans H− differing by a log order of magnitude is predictive of the likelihood of biofilm formation.

65. A method as claimed in claim 61 wherein a proportion of Acidovorax temperans H− to Acidovorax temperans H+ differing by a log order of magnitude is predictive of the likelihood of flocculation.

66. A method as claimed in claim 62 wherein a proportion of Acidovorax temperans H− to Acidovorax temperans H+ differing by a log order of magnitude is predictive of the likelihood of flocculation.

67. A method of maintaining viability of, and/or, promoting growth of, Acidovorax temperans H+ as claimed in claim 28 and/or Acidovorax temperans H− as claimed in claim 34 in a composition, including at least the steps of: (a) determining the NaCl concentration in the composition; and, where the concentration is above approximately 0.5%, (b) reducing it to below approximately 0.5%.

68. A method of maintaining viability of, and/or, promoting growth of, Acidovorax temperans H+ as claimed in claim 28 and/or Acidovorax temperans H− as claimed in claim 34 in a composition, including at least the steps of: (a) determining the NaCl concentration in the composition; and, where the concentration is above approximately 1%, (b) reducing it to below approximately 1%.
